(19) United States
(12) Patent Application Publication
(10) Pub. No.: US 2025/0360300 A1
(43) Pub. Date: Nov. 27, 2025

(54) Non-Disconnectable Luer Lock

(71) Applicant: Becton Dickinson and Company, Franklin Lakes, NJ (US)

(72) Inventors: Rahul Rajan Puthukkad, Bengaluru (IN); Shraya Sajjanar, Dharwad (IN); Deepak Udupa, Mangalore (IN)

(21) Appl. No.: 18/671,327

(22) Filed: May 22, 2024

(51) Int. Cl. A61M 39/10

(52) U.S. Cl. CPC A61M 39/1011; A61M 2039/1033

(57) ABSTRACT

A connector, including a connector body comprising a proximal region, a distal region, a longitudinal axis extending between the proximal region and the distal region, a sidewall extending between the proximal region and the distal region, a fluid channel extending from the proximal region to the distal region, and a threaded surface on at least a portion of the sidewall in the distal region; and a collar coupled to distal region, the collar configured to (i) deform to permit engagement between the threaded surface and a medical device, and (ii) resist axial movement of the medical device once engaged with the threaded surface.

### FIELD OF THE INVENTION

The present disclosure is directed to connectors for medical devices or assemblies that reduce the likelihood of inadvertent or unintentional disconnection.

### BACKGROUND OF THE INVENTION

Fluid connections are routinely implemented in medical settings between components or devices involving the preparation or delivery of fluids to a patient. Such connections may include one or more Luer lock connectors to engage a syringe, vial, container, or other fluid source to an intravenous line, device, or secondary container for fluid transfer. Unintentional disconnection of a fluid connection can be problematic or harmful for both patients and clinicians. For example, unintentional disconnection of a closed system transfer device involving hazardous drugs (e.g., for chemotherapy or the like) can pose substantial health risks to a clinician. Moreover, in certain healthcare environments, such as home care settings, there may be a higher risk of unintended disconnection due children, pets, or other non-healthcare practitioners in proximity to the fluid connections of a patient. Accordingly, the present disclosure provides connectors, assemblies, and components thereof that reduce the likelihood of an unintentional disconnection of a fluid connection.

### SUMMARY OF THE INVENTION

In accordance with one aspect, the present disclosure is directed to a connector, comprising a connector body comprising: a proximal region, a distal region, a longitudinal axis extending between the proximal region and the distal region, a sidewall extending between the proximal region and the distal region, a fluid channel extending from the proximal region to the distal region, and a threaded surface on at least a portion of the sidewall in the distal region; and a collar coupled to distal region, the collar configured to (i) deform to permit engagement between the threaded surface and a medical device, and (ii) resist axial movement of the medical device once engaged with the threaded surface.

In some non-limiting embodiments or aspects, the collar may be rotatable with respect to the connector body.

In some non-limiting embodiments or aspects, the collar may have a restricted axial range of movement in a distal direction with respect to the connector body.

In some non-limiting embodiments or aspects, the collar may include one or more retention elements, each retention element including a distal face and a proximal face, wherein the distal face is not parallel to the proximal face.

In some non-limiting embodiments or aspects, the distal face may form an angle with the longitudinal axis between 30 degrees and 45 degrees.

In some non-limiting embodiments or aspects, the proximal face may form an angle with the longitudinal axis between 70 degrees and 80 degrees.

In some non-limiting embodiments or aspects, the proximal face may form an angle with the distal face between 55 degrees and 80 degrees.

In some non-limiting embodiments or aspects, an inner surface of the sidewall in the distal region may define a first diameter, the one or more retention elements may include a plurality of retention elements radially spaced around the collar, and the plurality of retention elements may define a second diameter therebetween less than the first diameter.

In some non-limiting embodiments or aspects, the second diameter may be between 55% and 65% of the first diameter.

In some non-limiting embodiments or aspects, the collar may be constructed from polypropylene.

In accordance with one aspect, the present disclosure is directed to a medical assembly, comprising: the connector of claim 1; and the medical device threadably coupled to the connector.

In some non-limiting embodiments or aspects, the medical device may be a closed system transfer device.

In accordance with one aspect, the present disclosure is directed to a medical assembly, comprising: a connector comprising: a proximal region, a distal region, a longitudinal axis extending between the proximal region and the distal region, a sidewall extending between the proximal region and the distal region, a fluid channel extending from the proximal region to the distal region, a threaded surface on at least a portion of the sidewall in the distal region, and one or more retention elements coupled to the distal region; and a medical device comprising: an external threading configured to engage the threaded surface of the connector, a connecting tip configured to receive at least a portion of the fluid channel of the connector, and one or more locking elements configured to cooperatively engage the one or more retention elements to (i) permit rotational coupling of the connector to the medical device, and (ii) resist rotational decoupling of the connector and the medical device.

In some non-limiting embodiments or aspects, each retention element may include a distal face and a lateral face, wherein the distal face is not parallel to the lateral face.

In some non-limiting embodiments or aspects, the distal face may form an angle with the longitudinal axis between 100 degrees and 110 degrees.

In some non-limiting embodiments or aspects, the lateral face may form an angle with the longitudinal axis between 80 degrees and 90 degrees.

In some non-limiting embodiments or aspects, the lateral face may form an angle with the distal face between 80 degrees and 90 degrees.

In some non-limiting embodiments or aspects, each locking element may include a ramped face and a side face, and the ramped face may be configured to contact the distal face of at least one retention element to permit rotation between the connector and the medical device in a first direction.

In some non-limiting embodiments or aspects, the side face may be configured to contact the lateral face of at least one retention element to prevent rotation between the connector and the medical device in a second direction opposite the first direction.

In some non-limiting embodiments or aspects, the medical device may be a closed system transfer device.

Further examples of the present disclosure will now be described in the following numbered clauses.
Clause 1: A connector, comprising: a connector body comprising: a proximal region, a distal region, a longitudinal axis extending between the proximal region and the distal region, a sidewall extending between the proximal region and the distal region, a fluid channel extending from the proximal region to the distal region, and a threaded surface on at least a portion of the sidewall in the distal region; and a collar coupled to distal region, the collar configured to (i) deform to permit engagement between the threaded surface and a medical device, and (ii) resist axial movement of the medical device once engaged with the threaded surface.
Clause 2: The connector of clause 1, wherein the collar is rotatable with respect to the connector body.
Clause 3: The connector of clause 1 or clause 2, wherein the collar has a restricted axial range of movement in a distal direction with respect to the connector body.
Clause 4: The connector of any of clauses 1-3, wherein the collar includes one or more retention elements, each retention element including a distal face and a proximal face, wherein the distal face is not parallel to the proximal face.
Clause 5: The connector of clause 4, wherein the distal face forms an angle with the longitudinal axis between 30 degrees and 45 degrees.
Clause 6: The connector of clause 4 or clause 5, wherein the proximal face forms an angle with the longitudinal axis between 70 degrees and 80 degrees.
Clause 7: The connector of any of clauses 4-6, wherein the proximal face forms an angle with the distal face between 55 degrees and 80 degrees.
Clause 8: The connector of any of clauses 4-7, wherein an inner surface of the sidewall in the distal region defines a first diameter, wherein the one or more retention elements includes a plurality of retention elements radially spaced around the collar, and wherein the plurality of retention elements define a second diameter therebetween less than the first diameter.
Clause 9: The connector of clause 8, wherein the second diameter is between 55% and 65% of the first diameter.
Clause 10: The connector of any of clauses 1-9, wherein the collar is constructed from polypropylene.
Clause 11: A medical assembly, comprising: the connector of any of clauses 1-10; and the medical device threadably coupled to the connector.
Clause 12: The medical assembly of clause 11, wherein the medical device is a closed system transfer device.
Clause 13: A medical assembly, comprising: a connector comprising: a proximal region, a distal region, a longitudinal axis extending between the proximal region and the distal region, a sidewall extending between the proximal region and the distal region, a fluid channel extending from the proximal region to the distal region, a threaded surface on at least a portion of the sidewall in the distal region, and one or more retention elements coupled to the distal region; and a medical device comprising: an external threading configured to engage the threaded surface of the connector, a connecting tip configured to receive at least a portion of the fluid channel of the connector, and one or more locking elements configured to cooperatively engage the one or more retention elements to (i) permit rotational coupling of the connector to the medical device, and (ii) resist rotational decoupling of the connector and the medical device.
Clause 14: The medical assembly of clause 13, wherein each retention element including a distal face and a lateral face, wherein the distal face is not parallel to the lateral face.
Clause 15: The medical assembly of clause 14, wherein the distal face forms an angle with the longitudinal axis between 100 degrees and 110 degrees.
Clause 16: The medical assembly of clause 14 or clause 15, wherein the lateral face forms an angle with the longitudinal axis between 80 degrees and 90 degrees.
Clause 17: The medical assembly of any of clauses 14-16, wherein the lateral face forms an angle with the distal face between 80 degrees and 90 degrees.
Clause 18: The medical assembly of any of clauses 14-17, wherein each locking element includes a ramped face and a side face, wherein the ramped face is configured to contact the distal face of at least one retention element to permit rotation between the connector and the medical device in a first direction.
Clause 19: The medical assembly of clause 18, wherein the side face is configured to contact the lateral face of at least one retention element to prevent rotation between the connector and the medical device in a second direction opposite the first direction.
Clause 20: The medical assembly of any of clauses 13-19, wherein the medical device is a closed system transfer device.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a side view of an example of a connector constructed in accordance with the principles of the present invention.

FIG. 2 is a side cross-sectional view of the connector of FIG. 1.

FIG. 3 is a close-up side cross-sectional view of the connector of FIG. 1.

FIG. 4 is a side view of an assembly of the connector of FIG. 1 with an example of a medical device in accordance with the principles of the present invention.

FIG. 5 is a side cross-sectional view of the assembly of FIG. 4.

FIG. 6 is a close-up side cross-sectional view of the assembly of FIG. 1 in a first configuration in accordance with the principles of the present invention.

FIG. 7 is a close-up side cross-sectional view of the assembly of FIG. 1 in a second configuration in accordance with the principles of the present invention.

FIG. 8 is a side view of an assembly of an example of a connector with an example of a medical device in accordance with the principles of the present invention.

FIG. 9 is a closer side view of the connector of FIG. 8.

FIG. 10 is a perspective view of an example of a medical device with a plurality of locking elements thereon in accordance with the principles of the present invention.

FIG. 11 is a perspective view of an assembly of an example of a connector with an example of a medical device in accordance with the principles of the present invention.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the concept as it is oriented in the drawing figures. However, it is to be understood that the concept may assume various alternative variations, except where expressly specified to the contrary. It is also to be understood that the specific devices illustrated in the attached drawings, and described in the following specification, are simply exemplary embodiments of the concept. Hence, specific dimensions and other physical characteristics related to the embodiments disclosed herein are not to be considered as limiting.

The word “comprising” and “comprises”, and the like, does not exclude the presence of elements or steps other than those listed in any claim or the specification as a whole. In the present specification, “comprises” means “includes” and “comprising” means “including.”

As used herein, “at least one of” is synonymous with “one or more of.” For example, the phrase “at least one of A, B, or C” means any one of A, B, or C, or any combination of any two or more of A, B, or C. For example, “at least one of A, B, and C” includes A alone; or B alone; or C alone; or A and B; or A and C; or B and C; or all of A, B, and C.

The term “at least” is synonymous with “greater than or equal to.” The terms “first”, “second”, and the like are not intended to refer to any particular order or chronology, but refer to different conditions, properties, or elements. As used herein, the singular form of “a”, “an”, and “the” include plural referents unless the context clearly dictates otherwise.

All numbers used in the specification and claims are to be understood as being modified in all instances by the term “about”. By “about” is meant within plus or minus twenty-five percent of the stated value. However, this should not be considered as limiting to any analysis of the values under the doctrine of equivalents.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less. The ranges and/or ratios disclosed herein represent the average values over the specified range and/or ratio.

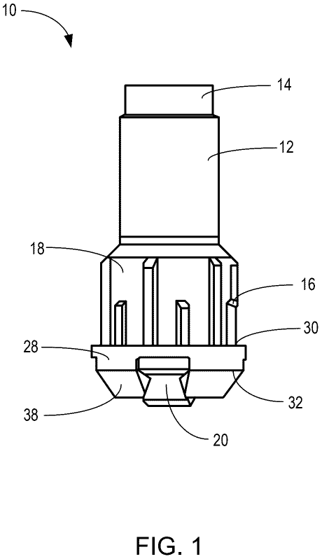

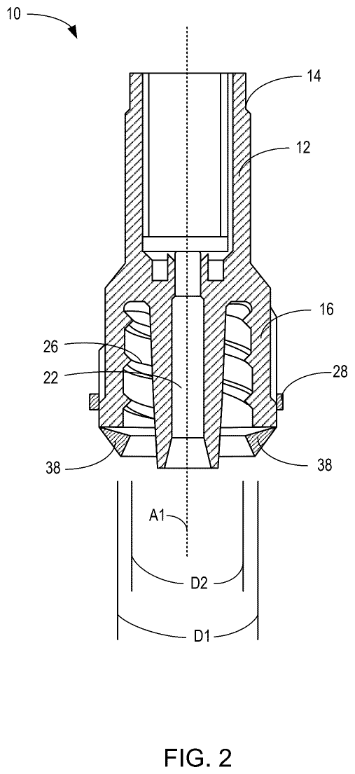

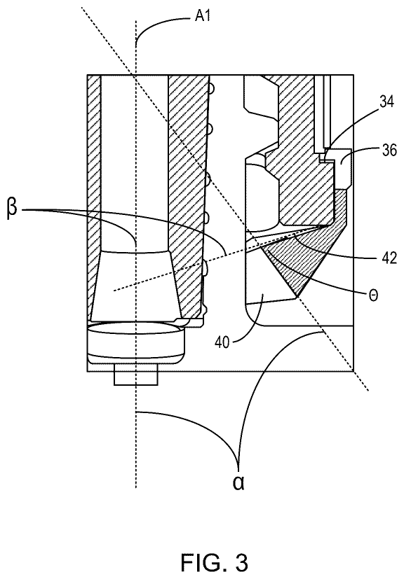

Reference is now made to FIGS. 1-3, which show a connector 10 configured to couple, engage, or otherwise connect two or more segments, components, and/or subassemblies of a medical system. The connector 10 may include a connector body 12 having a substantially cylindrical shape or form factor, and may include a luer lock connector and/or features thereof. The connector body 12 may generally define or include a proximal region 14, a distal region 16, and a sidewall 18 extending between the proximal and distal regions. The sidewall 18 may include one or more grip enhancing or tactile features to facilitate use as described herein. The connector 10 may include or define a longitudinal axis A1 extending between the proximal and distal regions.

The connector 10 may include or define a tip 20 at or adjacent to the distal region 16. The tip 20 may define or include a fluid channel 22 therein in fluid communication with a cavity 24 defined within at least a portion of the proximal region 14. The tip 20 may extend beyond a distal boundary or length of the sidewall 18, and may include a nozzle-shaped or tapered profile.

The connector 10 may define or include a threaded surface 26 on an interior portion of the sidewall 18 at, in, or adjacent to the distal region 16 configured to engage a complementary threaded surface on a medical device to be coupled to the connector 10. The proximal region 14 and/or cavity 24 may be configured to couple to and/or receive a fluid conduit, tubing, or other component of an assembly or device to be joined by the connector 10.

The connector 10 may include a collar 28 coupled to the connector body 12 and configured to prevent unintentional decoupling or disengagement of the connector 10 from an attached device. In one non-limiting example, the collar 28 may be configured to permit one-way rotational engagement of the threaded surface 26 with a device, but prevent, resist, or restrict unscrewing the threaded surface 26 from the attached device. In one non-limiting example, the collar 28 may be configured to permit one-way axial movement towards an attached device, but prevent, resist, or restrict axially displacing or moving the connector 10 away from the attached device.

The collar 28 may be attached, coupled, or otherwise positioned on or about an exterior surface of the sidewall 18 of the connector body 12. The collar 28 may be coaxially and/or concentrically attached, coupled, or otherwise positioned at, adjacent, or in proximity to the distal region 16. In one non-limiting example or aspect, the collar 28 may be rotatable with respect to the connector body 12. In one non-limiting example or aspect, the collar 28 may be axially movable towards the proximal region 14 of the connector body 12, and limited or restricted in a range of movement in a direction towards the distal region 14 of the connector body 12.

The collar 28 may define or include a proximal region 30 and a distal region 32. The proximal region 30 may define or include a substantially annular, ring-like, or cylindrically-shaped segment configured to mount or attach to the connector body 12. One or more engagement features may be included on or in the proximal region 30 of the collar 28 and/or the distal region 16 of the connector body 12 to facilitate or enable the connection and operation described herein. For example, an exterior portion of the sidewall 18 in the distal region 16 of the connector body 12 may include or define a shelf 34 providing an outwardly extending surface to support or couple to an inwardly-extending flange 36 on or about the proximal region 30 of the collar 28. The interaction of the flange 36 and the shelf 34 allows rotational movement between the collar 28 and the connector body 12, while preventing the collar 28 from sliding axially off the connector body 12 in a distal direction. Other engagement mechanisms may be implemented to provide the functions and features disclosed herein.

The collar 28 may include or define one or more retention elements 38 configured to prevent unintentional decoupling or disengagement of the connector 10 from an attached device. In one non-limiting example, the one or more retention elements 38 may be configured to permit one-way rotational engagement of the threaded surface 26 with a device, but prevent, resist, or restrict unscrewing the threaded surface 26 from the attached device. In one non-limiting example, the one or more retention elements 38 may be configured to permit one-way axial movement towards an attached device, but prevent, resist, or restrict axially displacing or moving the connector 10 away from the attached device. The one or more retention elements 38 may be radially spaced or circumferentially offset from one another around a perimeter or circumference of the collar 28.

In one non-limiting example or aspect, each of the one or more retention elements 38 may include or define a distal face 40 configured to contact and permit a portion of the medical device or component passing therethrough in a distal-to-proximal direction into an interior of the distal region 16 of the connector body 12. Each of the one or more retention elements 38 may include or define a proximal face 42 configured to prevent, resist, or reduce the ability of the coupled medical device or component to move in a proximal-to-distal direction to decouple from the connector 10.

The distal face 40 and proximal face 42 may have an angular orientation with respect to each other and/or the longitudinal axis A1 to provide the movement-permitting and restricting capabilities described herein. In a non-limiting example or aspect, the distal face 40 may be angled and/or positioned such that contact with an opposing thread or surface of a medical device to be received by the connector 10 results in a force vector with a radially outward component that radially displaces and/or deforms at least a portion of the collar 28 in a radial direction. In a non-limiting example or aspect, the proximal face 42 may be angled and/or positioned such that contact with an opposing thread or surface of a medical device attempting to decouple with the connector 10 results in a force vector with an axial component that is resisted at least in part by the engagement between the connector body 12 and the collar 28.

In one example, the distal face 40 may define an angle α between 30 degrees and 45degrees with respect to the longitudinal axis A1. The proximal face 42 may define an angle β between 70 degrees and 80 degrees with respect to the longitudinal axis A1. In one non-limiting example or aspect, the distal face 40 may define an angle θ between 55 degrees and 80 degrees with respect to the proximal face 42.

Each of the one or more retention elements 38 may extend inwardly past a radial position of an inner edge of the sidewall 18 in the distal region 16 of the connector body 12 to engage or contact a medical device passing into the interior of the distal region 16 to couple to the threaded surface 26. For example, an inner surface or edge of the sidewall 18 at the distal region 14 may define a diameter D1. The retention elements 38 may each define an inwardly positioned edge or surface that collectively defines a diameter D2 between opposing or spaced retention elements 38. In one example or aspect, the innermost surface or edge of each retention element 38 may include or define a connecting edge, corner, or intersection between the distal face 40 and the proximal face 42. The respective diameters D1 and D2 may be configured to facilitate and/or enhance the movement-permitting and restricting capabilities described herein in addition and/or alternatively to the angular orientations of the retention elements 38. In one example or aspect, D2 may be between 55% and 65% of D1. In one example or aspect, D1 may be between 10.5 mm and 11.75 mm. In one example or aspect, D2 may be between 6.3 mm and 6.8 mm.

The collar 28 may be constructed from one or more polymers, plastics, elastomers, and/or combinations thereof to provide sufficient flexibility to attach the collar 28 to the connector body 12 and to enable the retention elements 38 to deform and/or perform as described herein, while also providing sufficient strength to resist decoupling of a medical device from the connector 10, as also described herein. In some non-limiting examples or aspects, the collar 28 may be constructed from one or more of polypropylene.

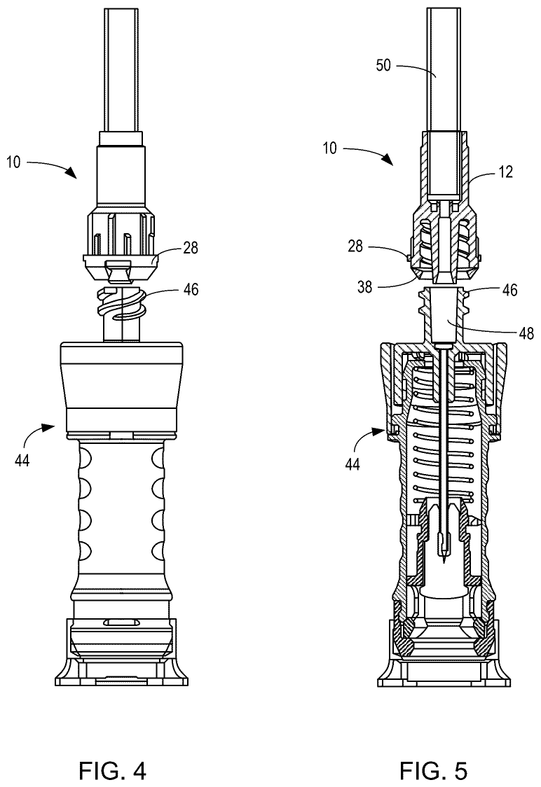

Now referring to FIGS. 4-7, an example of use of the connector 10 may include attaching the connector 10 to a medical device 44. The medical device 44 may include, for example, a closed system transfer device (as illustrated), an intravenous fluid line or device, or any other medical device, component, assembly configured to engage, accept, or receive a fluid connection. The medical device 44 may include or define an external threading 46 on or about a portion thereof configured to engage the threaded surface 26 of the connector body 12. The medical device 44 may define or include a cavity 48 configured to receive at least a portion of the tip 20 of the connector 10 to establish fluid communication between the connector 10 and the medical device 44. The connector 10 may be coupled to a fluid line or tube 50 at the proximal region 14 thereof to provide fluid communication through an assembly comprising the tube 50, connector 10, and the medical device 44. Referring to FIGS. 4-5, the connector 10 may be coaxially aligned with the medical device 44 with the retention elements 38 positioned axially adjacent the external threading 46. The connector 10 may be axially moved with respect to the medical device 44 such that the external 46 threading contacts the distal face 40 of one or more of the retention elements 38. Sufficient force may be applied between the distal face 40 and the external threading 46 to radially deflect and/or radially deform at least a portion of the one or more retention elements 38 and/or other portion of the collar 28 to permit the external threading 46 to pass through and/or by the retention elements 38. The external threading 46 may then engage and couple to the threaded surface 26 on the interior of the sidewall 18 of the connector body 12 through rotational engagement or suitable movement between the connector 10 and the medical device 44.

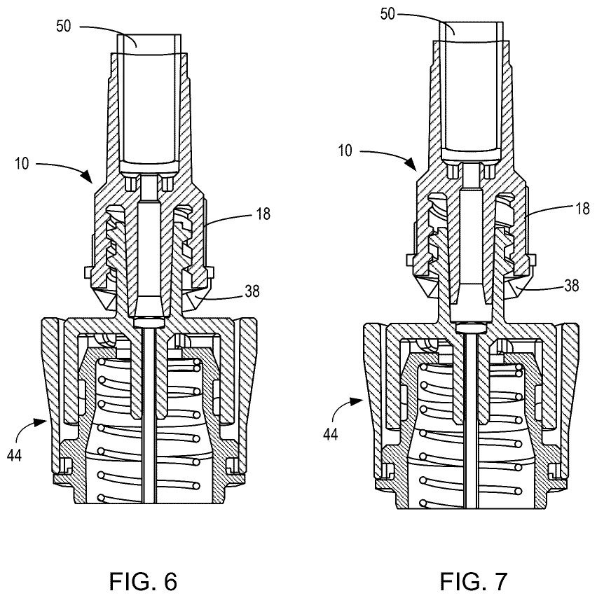

Referring now to FIG. 6, once the external threading 46 is axially past the one or more retention elements 38 and no longer contacting the distal faces 40 of the retention elements 38, the one or more retention elements 38 and/or the collar 28 may return to an original, un-deformed and/or non-deflected state. Referring now to FIG. 7, if the connector 10 and the medical device 44 experience a decoupling force through respective rotational and/or axial movement between the connector 10 and the medical device 44, the external threading 46 will contact the proximal face 42 of one or more of the retention elements 38, which restricts or resists further axial movement and thus disconnection between the connector 10 and the medical device 44.

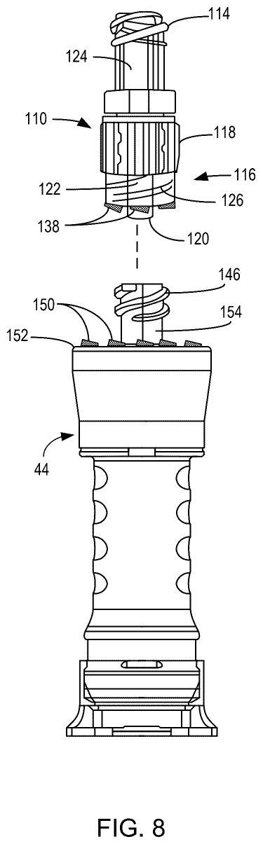

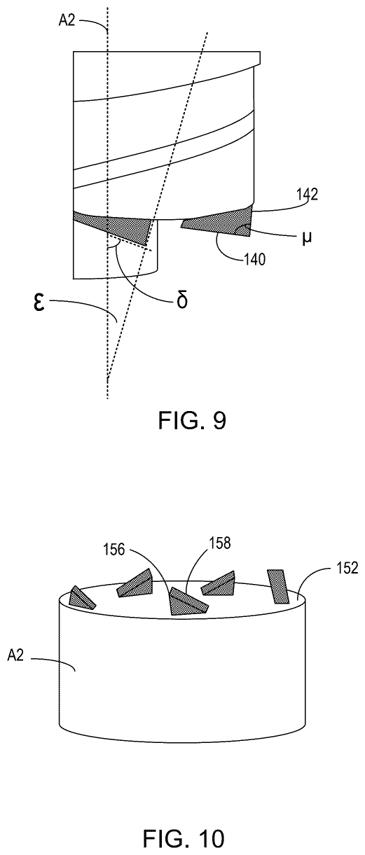

Now referring to FIGS. 8-9, another example of a connector 110 is provided that is configured to couple, engage, or otherwise connect two or more segments, components, and/or subassemblies of a medical system. The connector 110 may include features similar to those of connector 10 described herein, including a connector body 112 with a substantially cylindrical shape or form factor, and may include a luer lock connector and/or features thereof. The connector body 112 may generally define or include a proximal region 114, a distal region 116, and a sidewall 118 extending between the proximal and distal regions. The sidewall 118 may include one or more grip enhancing or tactile features to facilitate use as described herein. The connector 110 may include or define a longitudinal axis A2 extending between the proximal and distal regions.

The connector 110 may include or define a tip 120 at or adjacent to the distal region 116. The 1tip 20 may define or include a fluid channel 122 therein in fluid communication with a cavity 124 defined within at least a portion of the proximal region 114. The tip 120 may extend beyond a distal boundary or length of the sidewall 118, and may include a nozzle-shaped or tapered profile.

The connector 110 may define or include a threaded surface 126 on an interior portion of the sidewall 118 at, in, or adjacent to the distal region 116 configured to engage a complementary threaded surface on a medical device to be coupled to the connector 110. The proximal region 114 and/or cavity 124 may be configured to couple to and/or receive a fluid conduit, tubing, or other component of an assembly or device to be joined by the connector 110.

The connector 110 may include or define one or more retention elements 38 configured to prevent unintentional decoupling or disengagement of the connector 110 from an attached device. In one non-limiting example, the one or more retention elements 38 may be configured to permit one-way rotational engagement of the threaded surface 126 with a device, but prevent, resist, or restrict unscrewing the threaded surface 126 from the attached device. The one or more retention elements 138 may be radially spaced or circumferentially offset from one another around a perimeter or circumference of a distal-most face, surface, edge or other portion of the distal region 116 of the connector body 112.

In one non-limiting example or aspect, each of the one or more retention elements 138 may include or define a ramped or angled distal face 140 and a lateral face 142. In one example, the distal face 140 may define an angle δ between 100 degrees and 110 degrees with respect to the longitudinal axis A2. The lateral face 142 may define an angle ϵ between 80 degrees and 90 degrees with respect to the longitudinal axis A1. In one non-limiting example or aspect, the distal face 140 may define an angle μ between 80 degrees and 90 degrees with respect to the lateral face 142.

The connector 110 may be included in and/or comprise part of a kit or assembly with a medical device 144. The medical device 144 may include, for example, a closed system transfer device (as illustrated), an intravenous fluid line or device, or any other medical device, component, assembly configured to engage, accept, or receive a fluid connection. The medical device 144 may include or define an external threading 146 on or about a portion thereof configured to engage the threaded surface 126 of the connector body 112. The medical device 144 may define or include a cavity 148 configured to receive at least a portion of the tip 120 of the connector 110 to establish fluid communication between the connector 110 and the medical device 144. The connector 110 may be coupled to a fluid line or tube at the proximal region 114 thereof to provide fluid communication through an assembly including the connector 110 and the medical device 144.

The medical device 144 may include a plurality of locking elements 150 configured to cooperatively engage the retention elements 138 to resist or prevent decoupling of the connector 110 and the medical device 144 once they are securely engaged. The locking elements 150 may be substantial similar in size, shape, and/or configuration to the retention elements. In one non-limiting example or aspect, the one or more locking elements 150 may be radially spaced or circumferentially offset from one another around a perimeter or circumference of a surface, edge or other portion of the medical device 144 adjacent or in proximity to the external threading 146. For example, the medical device 144 may include or define a surface 152 at or near a base of a connecting tip 154 extending therefrom that includes the external threading 146 and at least a portion of the fluid channel 148.

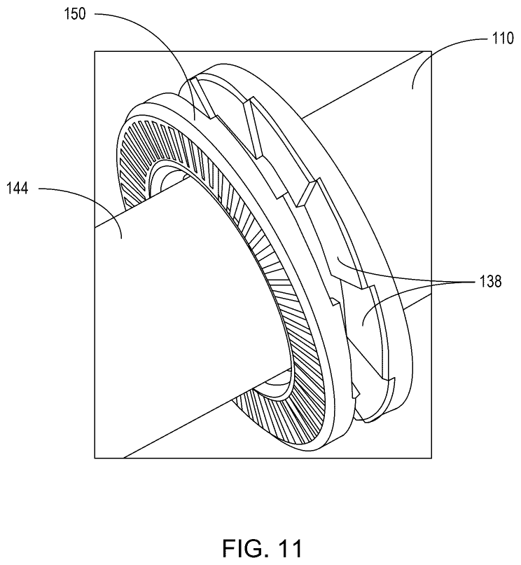

In one non-limiting example or aspect, each of the one or more locking elements 150 may include or define a ramped face 156 and a side face 158. The ramped face 156 may be sloped or angled to cooperatively engage or contact the distal face 140 of one or more retention elements 138 when the connector 110 is being threadably and/or rotatably coupled to the medical device 144. The side faces 158 of the locking elements 150 may be angularly oriented with respect to the lateral face 142 of each retention element 138 and/or the longitudinal axis to abut or otherwise contact the lateral face 142 of a retention element 138 to prevent rotation between the connector 110 and the medical device 144 in an opposite direction that would disengage the threaded engagement between the external threading 46 of the medical device 144 and the internal threaded surface 26 of the connector body 112. Non-limiting illustrative examples of angular features and configurations of the respective surfaces and faces of the retention elements 138 and the locking elements 150 are shown in FIGS. 10-11.

It will be appreciated by persons skilled in the art that the present disclosure is not limited to what has been particularly shown and described herein above. In addition, unless mention was made above to the contrary, it should be noted that all of the accompanying drawings are not to scale. Of note, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein. Moreover, while certain embodiments or figures described herein may illustrate features not expressly indicated on other figures or embodiments, it is understood that the features and components of the examples disclosed herein are not necessarily exclusive of each other and may be included in a variety of different combinations or configurations without departing from the scope and spirit of the disclosure. A variety of modifications and variations are possible in light of the above teachings without departing from the scope and spirit of the disclosure, which is limited only by the following claims.

What is claimed is:

1. A connector, comprising:
a connector body comprising:
a proximal region,
a distal region,
a longitudinal axis extending between the proximal region and the distal region,
a sidewall extending between the proximal region and the distal region,
a fluid channel extending from the proximal region to the distal region, and
a threaded surface on at least a portion of the sidewall in the distal region;
and
a collar coupled to distal region, the collar configured to (i) deform to permit engagement between the threaded surface and a medical device, and (ii) resist axial movement of the medical device once engaged with the threaded surface.

2. The connector of claim 1, wherein the collar is rotatable with respect to the connector body.

3. The connector of claim 1, wherein the collar has a restricted axial range of movement in a distal direction with respect to the connector body.

4. The connector of claim 1, wherein the collar includes one or more retention elements, each retention element including a distal face and a proximal face, wherein the distal face is not parallel to the proximal face.

5. The connector of claim 4, wherein the distal face forms an angle with the longitudinal axis between 30 degrees and 45 degrees.

6. The connector of claim 4, wherein the proximal face forms an angle with the longitudinal axis between 70 degrees and 80 degrees.

7. The connector of claim 4, wherein the proximal face forms an angle with the distal face between 55 degrees and 80 degrees.

8. The connector of claim 4, wherein an inner surface of the sidewall in the distal region defines a first diameter, wherein the one or more retention elements includes a plurality of retention elements radially spaced around the collar, and wherein the plurality of retention elements define a second diameter therebetween less than the first diameter.

9. The connector of claim 8, wherein the second diameter is between 55% and 65% of the first diameter.

10. The connector of claim 1, wherein the collar is constructed from polypropylene.

11. A medical assembly, comprising:
the connector of claim 1; and
the medical device threadably coupled to the connector.

12. The medical assembly of claim 11, wherein the medical device is a closed system transfer device.

13. A medical assembly, comprising:
a connector comprising:
a proximal region,
a distal region,
a longitudinal axis extending between the proximal region and the distal region,
a sidewall extending between the proximal region and the distal region,
a fluid channel extending from the proximal region to the distal region,
a threaded surface on at least a portion of the sidewall in the distal region, and
one or more retention elements coupled to the distal region; and
a medical device comprising:
an external threading configured to engage the threaded surface of the connector,
a connecting tip configured to receive at least a portion of the fluid channel of the connector, and
one or more locking elements configured to cooperatively engage the one or more retention elements to (i) permit rotational coupling of the connector to the medical device, and (ii) resist rotational decoupling of the connector and the medical device.

14. The medical assembly of claim 13, wherein each retention element including a distal face and a lateral face, wherein the distal face is not parallel to the lateral face.

15. The medical assembly of claim 14, wherein the distal face forms an angle with the longitudinal axis between 100 degrees and 110 degrees.

16. The medical assembly of claim 14, wherein the lateral face forms an angle with the longitudinal axis between 80 degrees and 90 degrees.

17. The medical assembly of claim 14, wherein the lateral face forms an angle with the distal face between 80 degrees and 90 degrees.

18. The medical assembly of claim 14, wherein each locking element includes a ramped face and a side face, wherein the ramped face is configured to contact the distal face of at least one retention element to permit rotation between the connector and the medical device in a first direction.

19. The medical assembly of claim 18, wherein the side face is configured to contact the lateral face of at least one retention element to prevent rotation between the connector and the medical device in a second direction opposite the first direction.

20. The medical assembly of claim 13. wherein the medical device is a closed system transfer device.
